(19) United States
(12) Patent Application Publication
(10) Pub. No.: US 2008/0026014 A1
(43) Pub. Date: Jan. 31, 2008

(54) Nasal Stick for the Application of Medicaments and Skin Care Substances

(76) Inventor: Olaf Michel, Koln (DE)

Correspondence Address: HARNESS, DICKEY & PIERCE, P.L.C., P.O. BOX 828, BLOOMFIELD HILLS, MI, 48303, US

(21) Appl. No.: 10/532,460

(22) PCT Filed: Oct. 31, 2003
(86) PCT No.: PCT/EP03/12150

§ 371 Date: May 19, 2006

(30) Foreign Application Priority Data
Oct. 31, 2002 (DE) 102 50 944.1

(51) Int. Cl. A61K 9/00; A61K 31/16; A61K 31/165; A61K 31/46; A61K 31/465; A61P 17/00; A61Q 19/00; A61K 31/522; A61K 31/573; A61K 38/11; A61K 47/44

(52) U.S. Cl. 424/401; 424/400; 424/489; 514/15; 514/181; 514/263.34; 514/304; 514/343; 514/625; 514/626; 514/772; 514/783; 514/786; 514/787

(57) ABSTRACT

A stick for the topical or transmucosal application of a skin care or active agent onto and via the nasal mucosa, respectively, is provided, wherein the stick material contains a composition of at least one carrier substance and at least one skin care or active agent, which is capable of acting on the nasal mucosa and/or being resorbed by it or which contains at least one volatile substance that acts on the deeper respiratory tract and/or is being resorbed by the deeper respiratory tract. Furthermore, the use of a medical skin care or cosmetical stick or of a stick material or of a refill cartridge containing said material for the topical or transmucosal application is described.

The invention relates to a stick for the topical or transmucosal application of a skin care or active agent onto and via the nasal mucosa, respectively, wherein the stick material contains a composition of at least one carrier substance and at least one skin care or active agent, which is capable of acting on the nasal mucosa and/or being resorbed by it or which contains at least one volatile substance that acts on the deeper respiratory tract and/or is being resorbed by the deeper respiratory tract. Furthermore, the invention relates to the use of a medical skin care or cosmetical stick or of a stick material or of a refill cartridge containing said material for topical or transmucosal application.

There are many advantages for the intranasal administration of medicaments and other preparations, for instance direct transport into the blood stream, avoiding the hepatic metabolism, bioavalability, simple and convenient administration and the physiologically close distance to the central nervous system; see also Chien et al., Nasal Systemic Drug Delivery, Chapter 1, (1989), 1-26. Different kinds of compositions, active agents, prophylactic agents and others can be administered intranasally, for instance topical anaesthetica, sedatives, analgetics, ketamines, opiates, glucagons, vaccines, antinause, laxatives and/or antidiarrhetic agents, antihistaminica, antihypertensives, psychoactive agents, antibiotics and hormones; see e.g., Nott et al., European Journal of Anaesthesiology, 12 (1995), 287-290; Henry et al., Journal of the American Academy of Pediatric Dentistry, 20 (1998), 321-326; Lithander et al., British Journal of Ophthalmology, 77 (1993), 529-530; Ralley, Intranasal Opiates: Old Route For New Drugs; Canadian Journal of Anaesthesiology, 36 (1989), 491-493, Haneberg et al., Infection and Immunity, 66 (1998), 1334-1341; Wagner et al., Pharmacotherapy, 16 (1996), 1063-1069.

The application of skin care and active agents onto the inner surface of the nose by sprays, ointments, powder or drops is known. These means of application have, however, the disadvantage that the active and skin care agents to be applied cannot be given in an exact dose nor can the retention time on the nasal mucosa be controlled. Often theses substances adhere to the nasal mucosa only for a short time, they tend to run off the nose due to unadjusted viscosity and therefore the retention time of the active agent on the nasal mucosa cannot be controlled and thereby no lasting effect of the skin care and active agent is possible.

Based on this, it is the object of the invention to provide a therapeutic system for the topical or transmucosal application of at least one skin care or active agent onto and via the nasal mucosa, respectively, as well as a method for the application of the system, which avoids the disadvantages mentioned above and allows a satisfying effect of the skin care and active agent(s) after application onto the nasal mucosa.

Said object is solved by the therapeutic system according to claim 1 by the topical or transmucosal application of a skin care or active agent onto and, respectively, via the nasal mucosa with a stick containing a stick material, which includes a composition of at least one carrier substance and at least one skin care or active agent, which is capable of acting on the nasal mucosa and/or being resorbed by it, or which contains at least one volatile substance that acts on the deeper respiratory tract and/or is being resorbed by the deeper respiratory tract.

Similar to a lip care stick, this stick is used for the application of active compounds into the nose. Apart from the disadvantages mentioned above of the application of compounds by sprays, ointments, drops or powder it is an advantage of the stick according to the invention and its use that the compounds can be applied to the vestibilum nasi only while introducing medicaments into the nose via a spray or with powder substances leads to their predominant adherence to inner surfaces of the nose that are located in the upper and backwards oriented parts. Furthermore, the use of said stick is much more convienient for a user since when applying sprays or drops, and particularly ointments and powders, he has to use his or her hands leading to soily or greasy fingers. The stick according to the invention however allows a clean application and can be manufactured furthermore in such a form which allows easy handling as well as decorative and functional design as for instance is common for lipsticks, due to the relatively solid consistency of the stick material. Thus, sticks according to the invention and the stick materials thereof, respectively, can be manufactured in principal using all common galenic basic materials as are used for cosmetical or skin care lipsticks, for example, vaselinum, algum, cocoa butter or soft paraffin. As mentioned before, the stick of the present invention is preferably solid at least at room temperature similar to lip sticks and in contrast to semi-solid gels that in some embodiments may be used, which however are generally less preferred.

From a technical viewpoint, most stick formulations are anhydrous fatty mixtures of solid or semisolid waxes and liquid oils, the highly pure paraffin oils and waxes forming the lipstick base material. Hydrous preparations are also known, which are sometimes also in the form of W/O emulsions. The ideal product profile includes smooth application of cosmetic or pharmaceutical sticks without substantial friction. Moreover, such a formulation must also satisfy the requirements that the stick in question must be resistant to breaking and to temperature and the formulation must not lose oil.

In particular, a stick even when pressed only lightly, should leave a non-greasy, non-dull or sticky, but nevertheless adherent lipid film For reasons of tolerance, it is always preferable, even when using substances which are acceptable per se, to keep the corresponding concentrations of such active ingredients as low as possible. Customary prior art base materials for stick preparations are, for example, liquid oils (e.g. paraffin oils, castor oil, isopropyl myristate), semisolid constituents (e.g. vaseline, lanolin), solid constituents (e.g. beeswax, ceresin and microcrystalline waxes or ozokerite) and high-melting waxes (e.g. carnauba wax, candelilla wax); see also the references cited herein.

The composition of the stick material of the stick according to the invention comprises essentially carrier substances, additives and/or adjuvants commonly used for cosmetical sticks and care sticks for example cocoa butter, waxes of vegetable and/or chemical origin, lipids of vegetable and/or chemical origin, Vaselinum algum, vegetable oils and/or petrochemical oils (paraffines), beeswax, shea butter, carnauba wax, micro-crystalline waxes, caprylic/capric triglycerides, lanolin, or mixtures of the above mentioned substances. The person skilled in the art is of course aware that demanding cosmetic preparations and preparations comprising active ingredient(s) are in most cases inconceivable without the customary adjuvant and additives. These include, for example, consistency regulators, fillers, perfume, dyes, emulsifiers, additional active ingredients such as vitamins or proteins, photoprotection agents, stabilisers, insect repellents, alcohol, water, salts, antimicrobial, proteolytic or keratolytic substances, etc.

In a preferred embodiment the composition of the stick material essentially contains cocoa butter and/or low melting olefines as carrier substances.

Preferred adjuvants and active ingredients are antioxidants, in particular those which are able to protect not only the constituents of the formulation, but also the skin against oxidative stress.

The preparations thus preferably comprise one or more antioxidants. Favourable, but nevertheless optional, antioxidants are all antioxidants which are suitable or customary for cosmetic and/or dermatological applications. It is preferred to use only a single class of antioxidants as active ingredient, for example when a cosmetic or dermatological use is of particular interest, such as the combating of oxidative stress of the skin.

The antioxidants are particularly preferred chosen from the group consisting of amino acids (for example glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. a-carotene, β-carotene, lycopene) and derivatives thereof, lipoic acid and derivatives thereof (e.g. dihydrolipoic acid), aurothioglucose, propylthiouracil and other thiols (e.g. thioglycerol, thiosorbitol, thioglycolic acid, thioredoxin, glutathione, cysteine, cystine, cystamine and the glycosyl, N-acetyl, methyl, ethyl, propyl, amyl, butyl and lauryl, palmitoyl, oleyl, ?-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulphoximine compounds (e.g. buthionine sulphoximines, homocysteine sulphoximine, buthionine sulphones, penta-, hexa-, heptathionine sulphoximine) in very small tolerated doses (e.g. pmol to μmol/kg), also (metal) chelating agents (e.g. a-hydroxy fatty acids, palmitic acid, phytic acid, lactoferrin), a-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives thereof (e.g. ascorbyl palmitates, Mg ascorbyl phosphates, ascorbyl acetates), isbascorbic acid and derivatives thereof, tocopherols and derivatives thereof (e.g. vitamin E acetate), vitamin A and derivatives thereof (e.g. vitamin A palnitate) and coniferylbenzoate of benzoin, rutinic acid and derivatives thereof, ferulic acid and derivatives thereof, butylated hydroxytoluene, butylated hydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO4), selenium and derivatives thereof (e.g. selenium methionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

For the purposes of the present invention, it is although particularly preferred to use oil-soluble or oil-dispersible antioxidants.

The amount of antioxidants (one or more compounds) in the preparations is preferably from 0.001 to 30% by weight, particularly preferably from 0.05 to 20% by weight, in particular from 1 to 10% by weight, based on the total weight of the preparation. If vitamin E and/or derivatives thereof are used as the antioxidant(s), it is preferred to choose their respective concentrations within the range from 0.001 to 10% by weight, based on the total weight of the preparation. If vitamin A or vitamin A derivatives, or carotenes or derivatives thereof are the antioxidant(s), it is preferred to choose their respective concentrations within the range from 0.001 to 10% by weight, based on the total weight of the preparation.

It is optionally possible and preferred to use the stick according to the invention as bases for pharmaceutical formulations. Corresponding requirements apply mutatis mutandis to the formulation of medicinal preparations. There is a smooth transition between pure cosmetics and pure pharmaceuticals. According to the invention, suitable pharmaceutical active ingredients are, in principle, all classes of active ingredients, with lipophilic active ingredients being preferred; see for general active agents for example German patent application P 21 40 050. Examples are: antihistamines, antiphlogistics, antibiotics, antimycotics, circulation-promoting active ingredients, keratolytics, hormones, steroids, vitamins, anti-inflammatory agents, anti-viral agents, anaesthetics, alkaloids, essential oils, aphrodisiaca, hallucinogenic drugs, nasal decongestives such as oxymetazoline, drugs affecting nitrogen monoxide production, agents for the treatment of allergies and/or colds such as menthol and eucalypt oil (see also DE 196 21 575 A1), local anaesthetics or (microencapsulated) liposomes and microsomes etc. Further examples of suitable pharmaceutical agents that can be applied with the stick in accordance with the present invention are described in the prior art; see, e.g., WO92/04019.

According to the invention, active ingredients are preferably chosen from the group consisting of lypophilic active ingredients, in particular from the following group: acetyl salicylic acid, atropine, azulene, hydrocortisone and derivatives thereof, e.g. hydrocortisone-17-valerate, vitamins, e.g. ascorbic acid and derivatives thereof, vitamins of the B and D groups, very favourably vitamin B1, vitamin B12, vitamin D1, but also bisabolol, unsaturated fatty acids, namely the essential fatty acids (often also called vitamin F), in particular d-linolenic acid, oleic acid, eicosapentanoic acid, docosahexanoic acid and derivatives thereof, chloramphenicol, caffeine, prostaglandins, thymol, camphor, extracts or other products of vegetable and animal origin, e.g. evening primrose oil, starflower oil or currant seed oil, fish oils, cod-liver oil or also ceramides and ceramide-like compounds etc.

In contrast, sticks comprising or consisting of silver nitrate, which are intended solely for the purpose of etching the nasal mucosa may not be encompassed by the present invention.

The amount of care or active ingredients (one or more compounds) in the preparations is preferably from 0.01 to 30% by weight, particularly preferably from 0.1 to 20% by weight, in particular from 1 to 10% by weight, based on the total weight of the preparation.

In a preferred embodiment of the stick according to the invention the active agent capable of being resorbed by the mucosa is selected from the group consisting of vasopressin, oxytocin, or a hormon such as sterol or derivates thereof, see also, e.g., DE 198 34 812 A1.

The local anaesthetic contained in the preparation is preferably xylocaine, pantocaine, cocain or another surface anaesthetic. Common alkaloids that can be used according to the invention include nicotine, coffeine, codeine, theophylline or theobromine.

Also the skin care properties of the stick according to the invention prove to be clearly improved compared to those of the prior art. For example, especially with compositions containing a high content of water even without further additives, a pleasant cooling effect on the skin can be achieved by mere application, which is pleasantly notable particularly in the case of hayfever.

The compositions may preferably include additional ingredients commonly used for skin care sticks, for example waxes, in particular, vegetable and/or animal waxes or chemically modified derivatives thereof, in particular carnauba wax, candelilla wax, sunflower wax, rice wax, fruit waxes such as orange wax, lemon wax, grapefruit wax, laurel wax (=Bayberry wax) and the like, hydrocarbons, lipids, oils, for the base, as well as the usual additives and adjuvants such as perfume oils, preservatives, dyes, photoprotection agents, stabilizers.

In addition, it is possible to incorporate skin care ingredients, which do not have to be limited to fat-soluble active ingredients as mentioned above, but can also be chosen from the group of water-soluble active ingredients, for example vitamins and the like. Preferably, the skin care ingredient is selected from a depilatory agent, dexpanthenol, a vitamin, cortison, sea salt or Ems salt; see also DE 34 40 844 A1.

In a particularly preferred embodiment of the stick according to the invention the stick material contains liposomes and/or fluids (e.g. in the form of filled nanoparticles). Advantageously the fluid contains at least one of above mentioned skin care and/or active agents.

In another embodiment the stick according to the invention contains additionally photoprotection agent(s) of SPF 1-26 in sufficient amounts. However, it is also preferred for the purposes of the present invention to provide cosmetic and dermatological compositions whose main purpose is not protection against sunlight, but which nevertheless still contain UV protection substances, for example to protect the formulation itself. Thus, for example, UV-A and UV-B filter agents are usually incorporated into day creams.

The sticks according to the invention are commonly of cylindrical, i.e. of circular shape as also known for lip care sticks and others. For example, DE 44 29 037 A1 discloses a stick, which consists of a base connected with the stick material and a sleeve surrounding said material. The driving device for moving the stick material in and out is provided as a injection moulded device in one piece together with said sleeve and base. According to the description of DE 44 29 037 A1 the sleeve may have a circular or an elliptical profile.

From DE 41 20 969 A1 a refill cartridge for the sleeve of the stick is known wherein the stick is used i.a. as glue-, antitranspirant-, lip or lip care stick. Such a refill cartridge is also disclosed in DE 41 16 581 C2. Furthermore, DE 91 17 141 U1 also shows a refill cartridge for the mentioned sticks. Such a system is also described in the DE 197 03 140 A1. A stick that is particularly capable of applying the stick material that is provided by said stick onto a surface in an areal form as well as a punctiform or line shaped manner is described in DE 199 54 004 A1.

All of these sticks and stick sleeves, respectively, may be used to provide a stick according to the invention. Thus, in principal also lip sticks or skin care sticks may be prepared or adapted in such a way that they can be applied according to the invention although they were not intended for introducing active agents into the nose as by their shape or their ingredients. Such sticks and their use, respectively, are also object of the present invention.

Important for the sticks according to the invention is merely that they are suitable for the application of skin care or active ingredients onto the vestibulum nasi. Preferably, said stick is designed such that the stick material has a profile at its upper end allowing a precise punctiform or line shaped application of the mentioned agents onto the vestibulum nasi. Accordingly, the stick and its stick material, respectively, are usually adjustable to the inner surface of the nose particularly the end providing said material, and have a semicircular to a tube or cone shaped form.

If a stick according to the prior art is suitable for the use according to the invention the present invention relates particularly to the use of such a medical, skin care or cosmetical stick and stick material, respectively, as described above, or a refill cartridge contained within, for the application of skin care or active agents into the nose. According to preferred embodiments of the stick according to the invention the respective sticks on the one hand and sleeves or covers on the other hand have sizes from 0.5 cm to 2 cm.

The sleeves or covers of the stick according to the invention may be decorated with motives of music and movies, comic figures, ornaments, paintings or pictures, respectively, to give them an attractive design for the user. Furthermore, it is preferred if the stick is provided with special product information relating to the respective active ingredient(s). It is furthermore preferred if the consumer information on the respective sleeves or covers, is provided by means of removable adhesive labels. This ensures that the consumer can remove the label with the consumer information from the sleeve or cover after studying it, such that the special ornaments and the design of the cover or sleeves is left unimpaired.

According to a further preferred embodiment of the stick according to the invention the stick is provided in a package that opens at the base of the stick. This ensures that the user will automatically hold the stick at its base when taking it out of the package such that the stick will not get deformed or modified any other way in its outer shape. The user does not need to touch the stick after opening the package according to the preferred embodiment of the stick according to the invention since the package of this embodiment is such that unrapping first leads only to the removal of the lower part of the package, e.g. by removing a plastic band sticking on the inner side of the package. The upper part of the package containing the stick is removed only when the stick has been introduced into its cover such that a soiling of the hands of the user is avoided.

Unless stated otherwise all amounts percentages or parts refer to the weight in particular to the total weight of the preparations or of the respective mixtures.

The disclosure of the above-cited documents from prior art are hereby included by reference into the present application, in particular regarding the preparation of stick materials, respective compositions, carrier substances, additives and adjuvants as well as the preparation of sleeves and their parts. These and other embodiments are disclosed to a person skilled in the art and encompassed by the description and the example of the present invention. Further literature concerning the above-mentioned active ingredients and agents, carrier substances, additives and adjuvants, stick devices and sleeves that may be used in the sense of the present invention can be retrieved from prior art, e.g. from public libraries using for instance electronic devices. Additionally, other public databases such as “Medline” available by the internet can be utilised.

The example below will serve to illustrate the invention without limiting it.

### EXAMPLE 1

Medical stick for the application of active ingredients into the nose characterised by its material having a composition consisting of
a) 50 to 99.9% cocoa butter
b) 1 to 35% waxes of vegetable or chemical origin
c) 0.1 to 35% additional lipids of vegetable or chemical origin
d) 0.001 to 5% vitamin E
e) 0.001 to 3% active ingredient
f) 0.001 to 3% antioxidant
g) ad. 100% vegetable oils and/or petrochemical oils (paraffins)

1. Stick for the topical or transmucosal application of a skin care or active agent onto and via the nasal mucosa, respectively, wherein the stick material contains a composition of at least one carrier substance and at least one skin care or active agent, which is capable of acting on the nasal mucosa and/or being resorbed by it or which contains at least one volatile substance, that acts on the deeper respiratory tract and/or is being resorbed by the deeper respiratory tract.

2. The stick of claim 1, wherein the composition comprises essentially carrier substances, additives and/or adjuvants commonly used for cosmetical sticks.

3. The stick of claim 1, wherein the carrier substances are selected from the group consisting of cocoa butter, waxes of vegetable and/or chemical origin, lipids of vegetable and/or chemical origin, Vaselinum algum, vegetable oils and/or petrochemical oils (paraffines), beeswax, shea butter, carnauba wax, micro-crystalline waxes, caprylic/capric triglycerides, lanolin, or mixtures of the above mentioned substances.

4. The stick of claim 1, wherein the composition essentially contains cocoa butter and/or low melting olefines as carrier substances.

5. The stick of claim 1, wherein the composition contains at least one antioxidant.

6. The stick of claim 1, wherein the active agent is selected from an anti-inflammatory agent, anti-viral agent, hormone, anaesthetic, alkaloid, antibiotic, essential oil, aphrodisiacum, hallucinogenic drug, nasal decongestive such as oxymetazoline, drug affecting nitrogen monoxide production, agent for the treatment of allergies and/or colds such as menthol and eucalypt oil, local anaesthetic or microencapsulated liposomes and microsomes.

7. The stick of claim 1, wherein the active agent capable of being resorbed by the mucosa is selected from oxytocin, vasopressin, or a hormone such as sterol or derivates thereof.

8. The stick of claim 1, wherein the care agent is selected from a depilatory agent, dexpanthenol, vitamin, cortisone, sea salt or Ems salt.

9. The stick of claim 6, wherein the local anaesthetic is xylocaine, pantocaine, cocaine or another surface anaesthetic.

10. The stick of claim 6, wherein the alkaloid is nicotine, caffeine, codeine, theophylline or theobromine.

11. Stick of claim 1, wherein the stick material contains liposomes and/or fluids (e.g. in the form of filled nanoparticles).

12. Stick of claim 11, wherein the nanoparticles contain at least one of above mentioned skin care and/or active agents.

13. Stick of claim 1, which additionally contains a preservative.

14. Stick of claim 1, which contains photoprotective agent(s) of SPF 1-26 in sufficient amounts.

15. Stick of claim 1, which is of cylindrical shape.

16. Stick of claim 15, which consists of a base connected with the stick material and a sleeve surrounding said substance.

17. Stick of claim 16, wherein the driving device for moving the stick material in and out is provided as a injection moulded device in one piece together with said sleeve and base.

18. Stick of claim 15, wherein said sleeve is provided with the stick material using a refill cartridge.

19. Stick of claim 1 suitable for delivering skin care or active agents to the vestibulum nasi.

20. Stick of claim 15, wherein the upper end of said stick material is shaped to allow a precise punctiform or lineshaped application of said agents onto the vestibulum nasi.

21. Stick of claim 1, wherein stick and sleeve, respectively, have a size from 0.5 to 2 cm.

22. Stick of claim 1, wherein the sleeve is decorated with motifs from music and movies, comic figures, ornaments, paintings or pictures.

23. Stick of claim 1, wherein said stick is provided with specific product information referring to said agents and their application.

24. Stick of claim 1, wherein said sleeve is provided with consumer information by means of a removable adhesive label.

25. Stick of claim 1, wherein said stick is provided in a package that opens at the base of the stick.

26. Use of a medical, skin care or cosmetical stick for the application of skin care or active agents into the nose.

27. Use of a stick material as defined in claim 1 or of a refill cartridge containing said material for the manufacture of a stick according to claim 1.

28. A method of treatment and/or prophylaxis of a disorder of a mammal comprising topical or transmucosal administration of an active agent as defined in claim 1 into the nose via a stick of claim 1.

29. A method of providing wellness or relief to a mammal comprising topical or transmucosal administration of an agent as defined in claim 1 into the nose via a stick of claim 1.

30. The method of claim 28, wherein said mammal is human.
